(19) United States
(12) Patent Application Publication
(10) Pub. No.: US 2006/0183671 A1
(43) Pub. Date: Aug. 17, 2006

(54) Treatment of anemia

(76) Inventors: Anne Angelillo-Scherrer, Vesenaz (CH); Peter Carmeliet, Oud-Heverlee (BE); Desire Collen, Winksele (BE)

Correspondence Address: CLARK & ELBING LLP, 101 FEDERAL STREET, BOSTON, MA, 02110, US

(21) Appl. No.: 10/595,044

(22) PCT Filed: Jul. 19, 2004
(86) PCT No.: PCT/BE04/00105

§ 371 Date: Apr. 7, 2006

Related U.S. Application Data
(60) Provisional application No. 60/487,905, filed on Jul. 17, 2003.
(60) Provisional application No. 60/547,842, filed on Feb. 26, 2004.

(51) Int. Cl. A61K 38/40; A61K 38/18

(52) U.S. Cl. 514/6; 514/12

(57) ABSTRACT

The present invention relates to a method for the treatment of anemia based on the administration of the product of growth arrest-specific gene 6 (Gas6), its mutants, variants, active derivatives and the physiological tolerated salts of said Gas6 derivatives or of a group of biologically active substances inducing a GAS6 expression or releasing action or of a group of compounds that activate the Sky, AxI or Mer receptor tyrosine kinases. Furthermore the invention relates to a pharmaceutically effective composition to treat anemia. The composition comprises a pharmaceutically effective amount of a product of growth arrestspecific gene 6 (Gas6), or its mutant, variant, active derivative or the physiological tolerated salts of said Gas6 derivative or a biologically active substance that induces GAS6 expression or GAS6 release or a substance that activates the Sky, AxI or Mer receptor tyrosine kinases.

### FIELD OF THE INVENTION

The present invention relates to formulations and methods for the treatment or prevention or cure of anemia in humans or mammals and for the manufacture of a medicament to treat a human or mammalian patient to prevent, reduce or cure anemia of said patient. In particular, the anemia can be induced by hemolysis or caused by or associated with various disorders such as chronic disease, chronic renal failure, aplastic anemia, hemolytic anemia, malignancies, endocrine deficiencies or can be caused by a specific treatment such as chemotherapy.

### BACKGROUND OF THE INVENTION

The term anemia refers to a reduction below normal in hemoglobin level or red blood cells in the blood. Anemia is a major cause of morbidity and mortality. It may result from a reduced rate of production or increased rate of destruction of red blood cells or their loss from the circulation by bleeding or from defective red blood cells as in sickle cell anemia. Although the cause of anemia can be a primary disorder of the production or survival of red blood cells, in many cases it is secondary to diseases of other systems (Weatherall & Provan (2000) Lancet 355, 1169-1175).

The mature red blood cell has two main functions. First, it must survive in the circulation for as long as possible, most of the time in vessels smaller than its own diameter. Second, it must both preserve its hemoglobin in state suitable for oxygen transport and adapt the amount of oxygen that is delivered to the needs of the tissues (Weatherall & Provan, cited above).

The process by which erythroid cells are produced is called erythropoiesis. The production of mature red blood cells from pluripotent hematopoietic stem cells requires the coordinated action of different cytokine signaling pathways to assure controlled cell proliferation, survival, differentiation and death. Under normal conditions, the whole process of erythropoiesis results in a red blood production rate such that the red cell mass in the body is kept onstant. Erythropoiesis involves a great variety and number of cells at different tages of maturation, starting with the first stem cell progeny committed to rythroid differentiation and ending with the mature circulating red blood cell. Erythropoiesis occurs in distinct stages and anatomic sites during development. Primitive erythropoiesis begins around embryonic day 7 when red blood cells originate in the blood islands of the yolk sac. These cells express embryonic globins and are not dependent upon erythropoietin (EPO)-[Wu et al. (1995) Cell 83, 59-67; Lin et al. (1996) Genes Dev. 10, 154-164]. EPO is the major cytokine for definitive and adult erythropoiesis. Definitive erythropoiesis starts at approximately day 10 when the site of red blood cell production shifts from the yolk sac to the foetal liver and is characterised by the production of non-nucleated erythrocytes expressing adult globin genes. At birth, erythroblastic islands in the bone marrow and the red pulp of the spleen become dominant sites of red blood cell production.

Epo also regulates the compensatory erythropoietic response to reduced tissue oxygen tension (hypoxia) and anemia. In humans, elevated Epo levels promote recovery from anemia by stimulating the generation of erythroid progenitors in the bone marrow and spleen. In the mouse, Epo enhances erythropoiesis primarily in the spleen.

Recombinant Epo is in widespread clinical use to treat anemia associated with a variety of disorders that include, for example, chronic renal failure, myelodysplastic syndrome, rheumatoid arthritis, and bone marrow transplantation, often resulting in substantial improvements in quality of life and prolonged survival (Winearls et al. (1986) Lancet 2(8517), 1175-1178.; Eschbach et al. (1987) N Engl J Med 316, 73-78). However, Epo is not uniformly effective, with many individuals being entirely refractory to even high doses (Horl et al. (2000) Nephrol Dial Transplant 15, 43-50). Over 50% of patients with cancer have an inadequate response to Epo, approximately 10% with end-stage renal disease are hyporesponsive (Glaspy et al. (1997) J Clin Oncol 15, 1218-1234; Demetri et al. (1998) J Clin Oncol 16, 3412-3425), and less than 10% with myelodysplastic syndrome respond favourably (Estey (2003) Curr Opin Hematol 10, 60-67). Although several factors, such as inflammation (Horl et al. (2000) cited above), iron and vitamin deficiency, inadequate dialysis, aluminium toxicity, and hyperparathyroidism (Macdougall (1995) Nephrol Dial Transplant 10, 607-614; Muirhead et al. (1995) Am J Kidney Dis 26, S1-24; Nitta et al. (2002) Acta Haematol 108, 168-170; Goicoechea et al. (1998) Kidney Int 54, 1337-1343) have been identified that predict for a poor therapeutic response, the molecular mechanisms of resistance to Epo remain largely obscure.

Moreover, adverse effects and contraindications for treatment with Epo have been determined. Over-treatment with the hormone in patients undergoing hemodyalysis has led to excessive increases in the hematocrit, leading to the aggravation of hypertension (sometimes leading to hypertensive encephalopathy and headaches) or thrombotic complications (Horl et al. (2000) Nephrol Dial Transplant 15, 51-55).

Gas6, the product of the growth arrest-specific gene 6 (gas6), is a new member of the vitamin K-dependent protein family (Manfioletti et al. (1993) Mol Cell Biol. 13, 49764985; Schneider et al. (1988) Cell 54, 787-793). Proteins belonging to this family are characterised by post-translational γ-carboxylation of certain glutamic acid residues by a carboxylase, using vitamin K as cofactor. The γ-carboxyglutamic acid (Gla)-containing module in prothrombin, coagulation factors VII, IX and X, protein C, protein S, protein Z and Gas6 allow these vitamin K-dependent proteins to bind to negatively charged phospholipid membranes (Dahlback (2000) Lancet 355, 1627-1632). Gas6 is structurally similar to protein S, but lacks a loop, crucial for the anticoagulant activity of protein S (Manfioletti et al. cited above). The latter is a cofactor for activated protein C, which inactivates the coagulation factors Va and VIIIa (Dahlback (1991) Thromb Haemost 66, 49-61). Genetic deficiency of protein S in man is one of the most severe inherited risk factors for thrombosis (Borgel et al. (1997) Thromb Haemost. 78, 351-356).

Apart from a Gla-domain-dependent interaction with phospholipid membranes (Nakano et al. (1997) J Biol Chem. 272, 29411-29414). Gas6 also binds as a ligand to the receptor tyrosine kinases AxI (Ark, Ufo, Tyro7), Sky (Rse, Tyro3, Dtk, Etk, Brt, Tif) and Mer (c-Mer, Eyk, Nyk) (Varnum et al. (1995) Nature 373, 623-626; Godowski et al. (1995) Cell 82, 355-358; Nagata et al. (1996) J Biol Chem. 271, 30022-30027; Crosier et al. 1997, Pathology 29, 131-135; Chen J et al. 1997, Oncogene 14, 2033-2039) by its carboxy-terminal globular G domains (Nagata (1996) cited above). It has been implicated in reversible cell growth arrest (Manfiofti et al. (1993) cited above; Schneider et al. 1988, cited above), survival (Goruppi et al 1996, Oncogene 12, 471-80), proliferation (Goruppi et al cited above, Li et al (1996) J Neurosci. 16, 2012-2019; Nakano et al. (1995) J Biol Chem. 270, 5702-5705), and cell adhesion (Nakano et al (1997) cited above; Fridell et al. (1998) J Biol Chem 273, 7123-7126, McCloskey et al (1997) J Biol Chem 272, 23285-23291). Mice with a triple deficiency of AxI, Sky and Mer are viable, but have not been reported to suffer spontaneous bleeding or thrombosis (Lu et al (1999) Nature 398, 723-8).

It has been shown that Gas6 deficient (Gas6^{-/-}) mice, generated by homologous recombination, are born at the expected mendelian frequency. Gas6^{+/-} and Gas6^{-/-} mice are viable, fertile, appear normal and show no obvious differences in size, weight or behaviour. No genotypic differences in litter size were observed. These mice appeared to be protected against thrombosis because of a platelet dysfunction (Angelillo-Scherrer et al (2001) Nat Med 7, 215-21).

Gas6 expression has been detected in hematopoietic tissue, both in hematopoietic (megakaryocytes and myelomonocytic precursors) and stromal (endothelial cells, fibroblasts, adipocytes) cells (Avanzi et al (1997) Exp Hematol 25, 1219-26). Gas6 receptor AxI is expressed in hematopoietic progenitors and bone marrow stromal cells, at low levels in monocytes, and in neoplastic cells of the myeloid lineage (Neubauer et al (1994) Blood 84, 1931-1941). AxI and Sky are detectable at sites of embryonic hematopoiesis (Crosier et al (1996) Exp Hematol 24, 318-323). However, Gas6 is considered to have no mitogenic effect in the hematopoietic system, and not affecting the proliferative stimuli exerted by other hematopoietic growth factors (Avanzi et al (1997), cited above). More recently, monolayers of Gas6 expressing fibroblasts were shown to support the generation of colony-forming units in culture. This hematopoietic support is not vitamin K-dependent and soluble recombinant Gas6 does not substitute for co-culturing the hematopoietic progenitors with Gas6 expressing fibroblasts (Dormady et al. (2000) Proc Natl Acad Sci USA. 97, 12260-12265).

WO 96/28548 describes Gas6 as the activator or the Rse and Mer receptor protein kinases and suggests the use of Gas6 for the enhancement of survival, proliferation or differentiation of any cell type expressing these proteins.

### SUMMARY OF THE INVENTION

An object of the present invention is to provide formulations and methods for the treatment or prevention or cure of anemia in humans or mammals and for the manufacture of a medicament to treat a human or mammalian patient to prevent, reduce or cure anemia of said patient. In particular, the anemia can be induced by hemolysis or caused by or associated with various disorders such as chronic disease, chronic renal failure, aplastic anemia, hemolytic anemia, malignancies, endocrine deficiencies or can be caused by a specific treatment such as chemotherapy.

The present invention is based on a first observation that growth arrest-specific gene 6 (Gas6) expression is required for the development of sufficient erythroid reserves and to ensure an adequate hematopoietic response to an anemic challenge in humans and/or mammals. Furthermore it was found that treatment with a Gas6 compound can provide protection against anemia induced by hemolysis.

The present invention relates to a new method for the treatment of anemia based on the administration of a Gas6 product, e.g. the gene product of the growth arrest-specific gene 6 (Gas6), its mutants, variants, active derivatives and the physiological tolerated salts of these Gas6 derivatives or of a group of biologically active substances inducing a Gas6 expression or releasing action or of a group of compounds that activate the Sky, AxI and/or Mer receptor tyrosine kinases.

Thus, one object of present invention is the use of a Gas6 compound, e.g. Gas6 protein or an analogue, mutant, variant or derivative thereof, or a physiologically tolerated salt of said Gas6 derivative for the treatment or prevention of anemia and for the manufacture of a medicament to treat a patient to prevent, reduce or cure anemia of said patient. Another object is the use of biologically active substances inducing a Gas6 expression or release for the treatment or prevention of anemia and for the manufacture of a medicament for the prevention or treatment of anemia. Biologically active substances supporting, provoking or inducing a Gas6 expression can be identified by several methods. On example is a vector comprising the Gas6 gene or its polymorphisms (with or without promoters) that can be vectored into relevant cells. Such vectors may include plasmids or viral vectors for example as well known from gene therapy. Another example is a monoclonal antibody which binds to the AxI and/or Tyr and/or Mer receptor and activates these. Immunological techniques or PCR can be used to detect increased expression of Gas6 in cell cultures or in test animals. Alternatively, reporter constructs comprising the Gas6 promoter region operably linked to a reporter gene (e.g. luciferase, GFP, LacZ or Chloramphenicol transferase) can be used for screening transfected cells or transfected test animals such as zebrafish or C. elegans.

The anemia can be caused by or associated with various disorders such as chronic disease, chronic renal failure, aplastic anemia, hemolytic anemia, malignancies, endocrine deficiencies or can be caused by a specific treatment such as chemotherapy.

An additional observation in the context of the present invention was that administration of a Gas6 compound, in dosages which provided the protective effect against anemia did not result in above-average hematocrit levels or other side-effects, such as polycythemia. The results obtained by the present inventors indicate that treatment with a Gas6 compound can correct anemia, but will do so only up to levels that are in the normal range, not leading to excessive hemoglobin levels as observed with Epo. This is an important advantage for all applications of a Gas6 compound in the treatment of anemia, but may be a critical factor for the treatment of anemia in patients susceptible to the adverse side-effects of Epo, such as hypertension or polycythemia.

Thus, a further object of the present invention is the use of a Gas6 compound, e.g. Gas6 protein or an analogue, mutant, variant or derivative thereof, or a physiologically tolerated salt of said Gas6 derivative for the treatment or prevention of anemia in patients for which treatment with Epo (or an analogue, mutant, variant or derivative thereof, or a physiologically tolerated salt of said EPO derivative) is contra-indicated and for the manufacture of a medicament for the prevention or treatment of anemia in patients for which treatment with Epo is contra-indicated.

The invention is furthermore based on the observation that in the absence of Gas6, response to Epo (or an analogue, mutant, variant or derivative thereof, or a physiologically tolerated salt of said EPO derivative) is sub-optimal and that administration of a Gas6 compound together with erythropoietin or an analogue, mutant, variant or derivative thereof, or a physiologically tolerated salt of said EPO derivative, results in a synergistic rescue effect on erythropoiesis. This indicates that that the level of Gas6 can be considered as a limiting factor for the effectiveness of Epo. Moreover, the present results indicate that upon combination of a Gas6 compound and Epo or an analogue, mutant, variant or derivative thereof, or a physiologically tolerated salt of said EPO derivative, the dose of Epo can be reduced to levels which would normally be considered as sub-optimal, thereby diminishing the likelihood of the development of adverse effects related to Epo.

Thus, a further object of the invention is the use of a Gas6 compound such as Gas6 protein or an analogue, mutant, variant or derivative thereof, or a physiologically tolerated salt of said Gas6 derivative for the treatment or prevention of anemia in patients non-responsive to Epo or for the manufacture of a medicament for the treatment or prevention of anemia in a patient non-responsive to Epo.

A further object of the present invention is the use of a Gas6 compound such as Gas6 protein or an analogue, mutant or derivative thereof, or a physiological tolerated salt of said Gas6 derivative, in combination with erythropoietin (or an analogue, mutant, variant or derivative thereof, or a physiologically tolerated salt of said EPO derivative) for the treatment or prevention of anemia or for the manufacture of an antianemic drug or an antianemic composition. These medicaments can be used to increase the survival rate of the patient suffering of anemia.

The invention further relates to a method of selection of a formulation of a pharmaceutical ratio of a Gas6 compound to Epo (or an analogue, mutant, variant or derivative thereof, or a physiologically tolerated salt of said EPO derivative), said method comprising: determining the minimal dose of Epo (or an analogue, mutant, variant or derivative thereof, or a physiologically tolerated salt of said EPO derivative) which does not induce adverse side-effects in a patient; and administering in addition to a) incremental doses of the Gas6 compound so as to determine which combined Epo/Gas6 compound dosage ensures maintenance of target hemoglobin levels in said patient, selecting the ratio in accordance with the determination of step b)

Alternatively, according to the present invention a method of selection of a formulation of a pharmaceutical ratio of a Gas6 compound to Epo (or an analogue, mutant, variant or derivative thereof, or a physiologically tolerated salt of said EPO derivative), is provided wherein said method comprises: determining independently, the minimal dose of Epo (or an analogue, mutant, variant or derivative thereof, or a physiologically tolerated salt of said EPO derivative) and Gas6 compound which ensures elevation of hemoglobin level to the target level in a patient, and administering a combination of the minimal dose of both Epo (or an analogue, mutant, variant or derivative thereof, or a physiologically tolerated salt of said EPO derivative) and Gas6 compound and gradually reducing the dosage of Epo (or an analogue, mutant, variant or derivative thereof, or a physiologically tolerated salt of said EPO derivative), so as to determine which combined Epo/Gas6 compound dosage ensures maintenance of target hemoglobin levels in said patient, without inducing the adverse side-effects of Epo.

A particular embodiment of this aspect of the invention relates to the use of a Gas6 compound, such as Gas6 protein or an analogue, mutant, variant or derivative thereof, or a physiologically tolerated salt of said Gas6 derivative in an optimized dosage combination with Epo (or an analogue, mutant, variant or derivative thereof, or a physiologically tolerated salt of said Epo derivative) for the treatment or prevention of anemia in a patient for which treatment with Epo is contra-indicated, or in a patient non-responsive to Epo. Similarly, the invention relates to the use of a Gas6 compound such as Gas6 protein or an analogue, mutant, variant or derivative thereof, or a physiologically tolerated salt of said Gas6 derivative for the manufacture of a medicament for the prevention or treatment of anemia in a patient for which treatment with Epo is contra-indicated, or in a patient non-responsive to Epo.

The invention further relates to method of augmenting the anti-anemic effect of erythropoietin (or an analogue, mutant, variant or derivative thereof, or a physiologically tolerated salt of said EPO derivative) being administered to a patient being afflicted with anemia, said method comprising administering to said patient an erythropoietin anti-anemia effect augmenting amount of a Gas6 compound in combination with a portion of an effective amount of erythropoietin (or an analogue, mutant, variant or derivative thereof, or a physiologically tolerated salt of said EPO derivative) normally required to treat anemia in said patient in the absence of said Gas6 compound, thereby decreasing the dosage of erythropoietin (or an analogue, mutant, variant or derivative thereof, or a physiologically tolerated salt of said EPO derivative) normally required to treat anemia in said patient to said portion of an effective amount and reducing the side effects caused by erythropoietin.

Optionally, the invention relates to the above-described method, wherein the amount of erythropoietin (or an analogue, mutant, variant or derivative thereof, or a physiologically tolerated salt of said EPO derivative) being administered is 5 to 90% of the amount of erythropoietin (or an analogue, mutant, variant or derivative thereof, or a physiologically tolerated salt of said EPO derivative) normally administered in the absence of said Gas6 compound.

Accordingly, a method is provided for the treatment, curing or prevention of anemia in a patient for which treatment with Epo is contra-indicated or in a patient who is irresponsive to Epo, which comprises administering thereto an optimized combination of a Gas6 compound such as Gas6 protein or an analogue, mutant or derivative thereof, or a physiologically tolerated salt of said Gas6 derivative, and erythropoietin, either simultaneously or sequentially.

According to another aspect of the present invention a method is provided for the treatment, curing or prevention of anemia, wherein the patient to be treated receives an effective amount biologically active substances inducing a Gas6 expression or releasing action and for a suitable time to prevent, reduce or cure anemia. This method may further comprise administering of erythropoietin (or an analogue, mutant, variant or derivative thereof, or a physiologically tolerated salt of said EPO derivative).

Yet another embodiment is an anti-anemic pharmaceutical composition comprising a Gas6 compound such as Gas6 protein or an analogue, mutant or derivative thereof, or a physiological tolerated salt of said Gas6 derivative or an optimized formulation of such a Gas6 compound with erythropoietin (or an analogue, mutant, variant or derivative thereof, or a physiologically tolerated salt of said EPO derivative).

### DESCRIPTION OF THE ILLUSTRATIVE EMBODIMENTS

The reference to Gas6, a mutant, variant or derivative thereof (generally referred to herein as Gas6 compounds) includes the Gas6 protein as encoded by the human Gas6 gene (Manfiotti et al. (1993) Molec. Cell Biol 13, 4976-4985), any proteins having a modified amino acid sequence, whereby this modification does not detrimentally affect the activity of Gas6 described herein. Reference to Gas6 or a Gas6 compound excludes EPO or an analogue, mutant, variant or derivative thereof, or a physiologically tolerated salt of said EPO derivative. The reference to Gas6 therefore includes species (human or animal) specific homologs or orthologs of Gas6 as well as truncations, deletions, point mutations, substitautions or other modifications made by man which maintain functionality. The maintenance of the activity of a Gas6 protein with a modified amino acid can be compared by determining for example the changes in hematocrite level of such modified protein, when compared to the wild type protein. Alternatively, reference to Gas6 function of a Gas6 compound may include proteins having a modified amino acid sequence, whereby this modification does not affect the activation of the Tyr and/or Mer and/or AxI receptor, the activation being of the type that is induced by wildtype Gas6. Activation of the Tyr or Mer receptor can be tested by assaying the tyrosine phosphorylation by Tyr or Mer of a substrate peptide. As used herein “mutants of Gas6” refers to modified Gas6 proteins of equal length as wild type Gas6 but with one or more modified amino acids. The sequence homology of such modified Gas6 proteins have a homology of 95% or greater, or 98% or greater or 99% or greater compared to the wildtype Gas6. As used herein “variants” refers to modified Gas6 proteins with a length differing from wild type Gas6 obtained by removal or addition of one or more amino acids (either N-terminal, 1° C.-terminal or internally, for instance the variants which are less or not γ-carboxylated, fragments of Gas6 which lack the A domain as well as fragments which consist essentially of the D domain, such as those described in WO 96/28548. The sequence identity of such variant Gas6 proteins can be 70% or greater compared to the wildtype Gas6.

A Gas6 analogue as used herein refers to a molecule capable of activating the Tyr, Mer and/or AxI receptor on hematopoietic cells, in a similar way as Gas6. These molecules can optionally be directly or indirectly derived from Gas6. Protein S has been reported to be a ligand for Tyro3, which is a receptor of the Tyr, Mer and AxI family and which binds also Gas6. Gas6 analogues can have a structure being related to Gas6 (peptide analogues) or unrelated to Gas6 such as small compounds. Gas6 analogues can be screened by evaluating the activation of a Gas6 receptor by assaying for example the tyrosine kinase activity of such receptor on a substrate peptide.

A physiologically tolerated salt of Gas6, or a mutant, variant or derivative thereof refers to any therapeutically active non-toxic salts which the Gas6 compounds are able to form with a salt-forming agent. Such addition salts may conveniently be obtained by treating the Gas6 compounds of the invention with an appropriate salt-forming acid or base. Examples of such appropriate salt-forming acids include, for instance, inorganic acids resulting in forming salts such as but not limited to the hydrochloride, hydrobromide, sulfate, nitrate, phosphate, diphosphate, bicarbonate, carbonate salts, and the like; or organic monocarboxylic or dicarboxylic acids resulting in forming salts such as, for example, the acetate, propanoate, hydroxyacetate, 2-hydroxypropanoate, 2-oxopropanoate, lactate, pyruvate, oxalate, malonate, succinate, maleate, fumarate, malate, tartrate, citrate, methanesulfonate, ethanesulfonate, benzoate, benzenesulfonate, p-toluene-sulfonate, salicylate, p-aminosalicylate, pamoate, bitartrate, camphorsulfonate, edetate, 1,2-ethanedisulfonate, fumarate, glucoheptonate, gluconate, glutamate, hexylresorcinate, hydroxynaphtoate, hydroxyethanesulfonate, lactate, mandelate, methylsulfate, pantothenate, stearate and the like. Examples of appropriate salt-forming bases include, for instance, inorganic bases like metallic hydroxides such as but not limited to those of calcium, lithium, magnesium, potassium, sodium and zinc; organic bases such as but not limited to N,N′-dibenzylethylenediamine, chloroprocaine, choline, diethanolamine, ethylene-diamine, N-methylglucamine, procaine and the like.

Erythropoietin or Epo as used herein refers to the naturally occurring human cytokine, produced primarily in the kidney which stimulates the production of red blood cells, as well as analogues, mutants, variants or derivatives thereof, or a physiologically tolerated salt of said EPO derivative. Well described erythropoietin analogues are the hyperglycosylated recombinant proteins epoetin and darbepoetin (or novel erythropoiesis stimulating protein, NESP), of which the structures differ from naturally occurring Epo only by the number of N-linked oligosaccharide on the protein. The invention however also envisages the possible development of other activators of the Epo receptor, which if developed into Epo analogues, can be used in the context of the present invention. The expression product of deletion mutants of a synthetic human Epo cDNA, wherein the point mutations and small deletions in helices and interhelical regions of the four alpha heleical bundle motif have been shown to display biological activity (Bittorf et al. (1993) FEBS Left 336, 133-136; Boissel et al. (1993) J. Biol. Chem. 268, 15983-15993). Furthermore, the screening of combinatorial libraries of dimeric iminodiacetic acid diamides, small molecule binders of the Epo receptor have been identified, of which some were found to be partial agonists (Goldberg et al. (2002) J Am Chem Soc 124, 544-555) In addition Johnson et al (1998) Biochemistry 37, 3699-3710 present amino acid peptide mimetics of EPO which were selected by phage display. US patent application US2003/0113871 discloses EPO fusion proteins with increased half-life. US patent application 2003/0077753 describes EPO variants with modified glycosylation patterns.

Anemia as used herein refers to a condition in which the number of red blood cells per mm³, the amount of hemoglobin in 100 ml blood and/or the volume of packed red blood cells per 100 ml blood are less than normal. When observing hemoglobin levels, a level of less than 9 g/dl is usually considered as indicative of anemia, though individual variations should be taken into account (see below). Potential causes include blood-loss, nutritional deficits, medication reaction, various problems with the bone marrow and many diseases. More particularly, anemia has been associated with a variety of disorders that include, for example, chronic renal failure, myelodysplastic syndrome, rheumatoid arthritis, and bone marrow transplantation. It will be understood that in the is context of the present invention, the anemic conditions susceptible to treatment with Gas6 optionally in combination with Epo exclude those conditions where anemia can be attributed to the absence of nutrients, such as vitamin BI 2 deficiency. More particularly, the use of Gas6, optionally in combination with Epo is envisaged in the treatment and/or prevention of the following disorders and conditions:

Anemia that is associated with malignant disease, including:

solid tumors (e.g. breast cancer, lung cancer, colon cancer)

tumors of the lymphatic system (e.g. chronic lymphocyte leukemia, non-Hodgkins and Hodgkins lymphomas)

tumors of the hematopoeitic system (eg. leukemia, myelodysplastic syndrome, multiple myeloma).

Anemia associated with radiation therapy.

Anemia associated with chemotherapy (e.g. platinum containing regimens) for cancers.

Anemia associated with inflammatory and autoimmune diseases, including, but not limited to, rheumatoid arthritis, other inflammatory arthritides, systemic lupus (SLE), acute or chronic skin diseases (e.g. psoriasis), inflammatory bowel disease (e.g. Crohn's and Ulcerative Colitis).

Anemia associated with acute or chronic renal disease or failure due to any cause, including those not defined, i.e. idiopathic or congenital.

Anemia associated with acute or chronic liver disease.

Anemia associated with acute or chronic bleeding.

Anemia for any reason where transfusion of red blood cells is not possible due to patient allo- or auto-antibodies and/or for religious reasons (e.g. some Jehovah's Witnesses).

Anemia associated with infections (e.g. malaria, osteomyelitis)

Anemia associated with hemoglobinopathies, including, for example, sickle cell disease, thalassemias.

Anemia associated with any chronic disease.

Anemia associated with drug use or abuse, e.g. alcohol misuse.

Pediatric patients with anemia from any cause to avoid transfusion.

Elderly patients or patients with underlying cardiopulmonary disease with anemia who cannot receive transfusions due to concerns about circulatory overload.

Patients who are iron-overloaded, yet require raised hemoglobin. These would include all patients with ineffective erythropoiesis of any cause.

The ‘target hemoglobin level’ as referred to herein is considered a hemoglobin level of between about 10 g/dl and about 12.5 g/dl preferably about 11.0 g/dl (see also Jacobs et al. (2000) Nephrol Dial Transplant 15, 15-19). Hemoglobin levels in healthy individuals (‘normal hemoglobin level’) are usually significantly higher (14-17.4 g/dl for males and 12.3-15.3 g/dl for females; Wintrobe MM, Clinical hematology, 10^{th} Ed Philadelphia.), without necessarily resulting in any clinical symptoms. Alternatively, hematocrit levels (percentage of the volume of a blood sample occupied by the cells, can be used as a measure for the condition of red blood cells. Hematocrit levels for healthy individuals range from 41 to 51 for adult males and from 35 to 45 for adult females. Target hematocrit levels are usually around 30-33%. It has been reported that upon obtaining a normal Ht (42.0±1.9%) in hemodialysis patients, both physiological tests and quality of life improved significantly without adverse effects (Minetti (1997) J Nephrol 10, 117-119). Thus, in the absence of contraindications (e.g. high cardiovascular risk), higher hemoglobin/hematocrit target levels can be contemplated (‘normalized hemoglobin level’), as long as adverse effects are not observed or anticipated. Moreover, it is understood to the person skilled in the art that hemoglobin/hematocrit levels vary from person to person. Thus, optimally, the target hemoglobin/hematocrit level should be individualized for each patient (Jacobs et al., cited above). ‘Overshooting’ or an excessive hemoglobin level as used herein, refers in a situation whereby a level of hemoglobin above 13 g/dL or higher than the normalized hemoglobin level is associated with adverse side-effects, such as hypertension and/or polycythemia.

A patient with an “inadequate Epo response” as used herein refers to a patient in which administering of a normal to increased (>300 IU/kg/week) dose of Epo does not result in the increase of haemoglobin level up to the target level. Optionally, in patients susceptible to adverse effects of Epo, the absence of a response to standard dosage of Epo (around 50-100 U/kg weekly) can be considered as “inadequate response to a tolerable dose of Epo”. Patients with an inadequate Epo response are found for all types of anemia, but higher numbers of non-responders have been observed particularly frequently in patients with cancers and patients with end-stage renal disease.

An inadequate response to Epo can be either constitutive (i.e. observed upon the first treatment with Epo) or acquired (e.g. observed upon repeated treatment with Epo). Thus, patients who do not show an initial positive response (i.e. reaching of target hemoglobin level) to Epo are considered “irresponsive to treatment with Epo” in the context of the present invention.

When an initial response to treatment with Epo is observed, but the response “resistant to Epo” refers to patients in which, upon prolonged and/or repeated treatment with Epo, there is a decreased response to exogenous Epo. This resistance has been linked to iron or folate deficiency, aluminum toxicity, hyperparathyroidism or auto-antibodies. In the case of an immune reaction to exogenous Epo the immune reaction can sometimes be ovecome with anti-CD4+ mAB administration (Rinsch et al. (2002) Kidney Intern. 62, 1395-1401).

A subset of patients with an inadequate Epo response is formed by those patients for which an elevated endogenous Epo level is observed. The absolute concentration of Epo considered as “elevated” is dependent on the disease and dosage of Epo. It has been observed that in Epo treatment of anemia associated with HIV infection and zodovudine therapy (100-200 U/kg, three times a week for up to 12 weeks), patients with Epo levels greater than 5001 U/L showed no benefit from recombinant Epo.

An “adverse effect of Epo” as used herein refers to effects which are associated with the Epo treatment and are unwanted. The primary adverse effects of Epo are an excessive increase in the hematocrit or hemoglobin levels and polycythemia. Elevated hematocrit levels can lead to hypertension (more particularly aggravation of hypertension) and vascular thrombosis. Other adverse effects of Epo which have been reported, some of which related to hypertension, are headaches, influenza-like syndrome, obstruction of shunts, myocardial infarctions and cerebral convulsions due to thrombosis, hypertensive encephalopathy, and red cell blood cell applasia (Singibarti, (1994) J. Clin Investig 72(suppl 6), S36-S43; Horl et al. (2000) Nephrol Dial Transplant 15(suppl 4), 51-56; Delanty et al. (1997) Neurology 49, 686-689; Bunn (2002) N Engl J Med 346(7), 522-523).

A patient “susceptible to the adverse effect of Epo” or “for which treatment with Epo is contra-indicated” as used herein refers to a patient for whom, due to natural (i.e. genetic) predisposition or due to other factors (e.g. disease, medication etc.), the adverse side effects of Epo would be more detrimental than for a person not having this natural or unnatural predisposition. More particularly, patients with increased blood pressure, either as a result of a genetic predisposition or as a result of vascular disease or medication, are considered more susceptible to the aggravation of hypertension observed in the treatment with Epo.

The present invention relates to the treatment and/or prevention of anemia with an effective amount of a Gas6 compound. An effective amount of a Gas6 compound to be employed therapeutically will depend, for example, on the therapeutive objections, the route of administration, and the condition of the patient. Accordingly it will be necessary for the therapist to titer the dosage and modify the route of administration as required to obtain the optimal therapeutic effect. The effective amount is usually in the range of 0.001 mg to 100 mg, preferably 0.1 mg to 5 mg, per day per kg bodyweight for humans. Depending upon the pathologic condition to be treated and the patient's condition, the said effective amount may be divided into several sub-units per day or may be administered at more than one-day intervals.

Where possible, it is desirable to determine appropriate dosage ranges first in vitro, for example, using assays for cell survival or growth, which are known in the art, and then in suitable animal models, from which dosage ranges for human patients may be extrapolated. In a specific embodiment of the invention, a pharmaceutical composition effective in stimulating hemoglobin production by hematopoietic cells will provide a local gas6 concentration in vivo of between about 0.1 and 10 ng/ml.

Alternatively, delivery of Gas6 and/or erythropoietin in the context of the present invention can be performed using gene therapy. Therapeutic levels of Epo using gene therapy has been described in the art, including in WO 95/13376 and EP 1105506. Similar technology can be applied for the administration of Gas6. Thus, for example, cells from a patient may be engineered with a polynucleotide (DNA or RNA) encoding a Gas6 polypeptide, a mutant, variant or derivative thereof or a Gas6 analogue ex vivo, with the engineered cells then being returned to said patient. Such methods are well-known in the art. For example, cells may be engineered by procedures known in the art using a retroviral or lentiviral particle containing RNA encoding a polypeptide of the present invention. Cells suitable for such cell-based gene therapy include, but are not limited to stem cells, sertoli cells, and include the use of encapsulated xenogeic cells. Alternatively, vectors (such as infectious viral particles or non-viral particles such as liposomes or micelles) comprising a DNA encoding a Gas6 polypeptide, a mutant, variant or derivative thereof or a Gas6 analogue, can be used for the engineering of Gas6 expressing cells in vivo.

The present invention further relates to the treatment and/or prevention of anemia with an optimized combination of Gas6 and Epo. Establishing an optimal combination of Epo and Gas6 requires titration of the two components, each adjusted to achieve a normal hemoglobin level, without “overshooting” the normal or target range, followed by a combined treatment with increasingly reduced dosage, so as to determine which minimal dosage can ensure the target or normalized hemoglobin level without excessive hemoglobin production and without adverse side effects.

The optimal combination of Gas6 and Epo can optionally be determined for every individual patient, so as to take into account individual susceptibility to adverse effects of Epo or Gas6 and/or individual irresponsiveness to Epo and/or Gas6.

The pharmaceutical compositions and combined preparations according to this invention may be administered orally or in any other suitable fashion. Oral administration is preferred and the preparation may have the form of a tablet, aqueous dispersion, dispersable powder or granule, emulsion, hard or soft capsule, syrup, elixir or gel. The dosing forms may be prepared using any method known in the art for manufacturing these pharmaceutical compositions and may comprise as additives sweeteners, flavouring agents, colouring agents, preservatives and the like. Carrier materials and excipients are detailed hereinbelow and may include, inter alia, calcium carbonate, sodium carbonate, lactose, calcium phosphate or sodium phosphate; granulating and disintegrating agents, binding agents and the like. The pharmaceutical composition or combined preparation may be included in a gelatine capsule mixed with any inert solid diluent or carrier material, or has the form of a soft gelatine capsule, in which the ingredient is mixed with a water or oil medium. Aqueous dispersions may comprise the biologically active composition or combined preparation in combination with a suspending agent, dispersing agent or wetting agent. Oil dispersions may comprise suspending agents such as a vegetable oil. Rectal administration is also applicable, for instance in the form of suppositories or gels. Injection is also applicable as a mode of administration, for instance in the form of injectable solutions or dispersions.

The term “pharmaceutically acceptable carrier or excipient” as used herein in relation to pharmaceutical compositions and combined preparations means any material or substance with which the biologically-active ingredient(s), i.e. the Gas6, or an analogue, mutant or derivative thereof, or a physiologically tolerated salt of said Gas6 derivative, may be formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, pellets or powders. Suitable pharmaceutical carriers for use in the said pharmaceutical compositions and their formulation are well known to those skilled in the art.

The present invention will be demonstrated in more detail in the following examples, which are however not intended to limit the scope of the invention, and may be understood in conjunction with the accompanying Figures, which show:

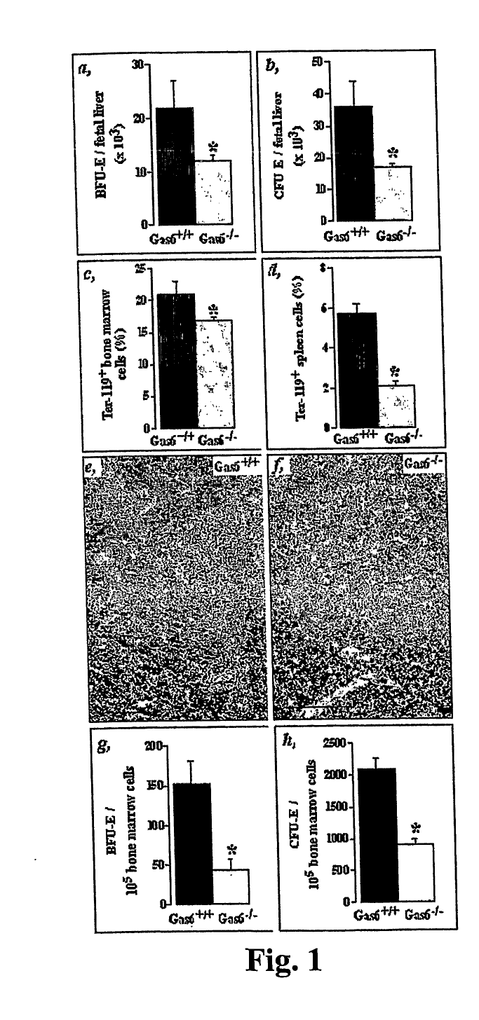

FIG. 1: a,b, Reduced number of erythroid progenitors in fetal liver of Gas6−/− mice, analyzed as the number of BFU-E (n=6; mean±S.E.M.; *, P<0.05; panel a) or CFU-E (n=6; mean±S.E.M.; *, P<0.05; panel b). c,d, Reduced percentage of Ter-119+nucleated cells (erythroblasts) in adult bone marrow (n=9; mean±S.E.M.; *, P=0.02) and spleen (n=9; mean±S.E.M.; *, P<0.02) in Gas6−/− mice. The erythroblasts were quantified as % of all nuceated cells by flow cytometry. e,f, Immunohistochemistry of adult WT (e) and Gas6−/− (f) spleen, showing fewer brown-stained Ter-119+ cells in Gas6−/− mice. g,h, Reduced number of erythroid progenitors in the bone marrow of adult Gas6−/− mice, analyzed as the number of BFU-E (n=8; mean±S.E.M.; *, P<0.05; panel g) or CFU-E (n=8; mean±S.E.M.; *, P<0.05; panel h).

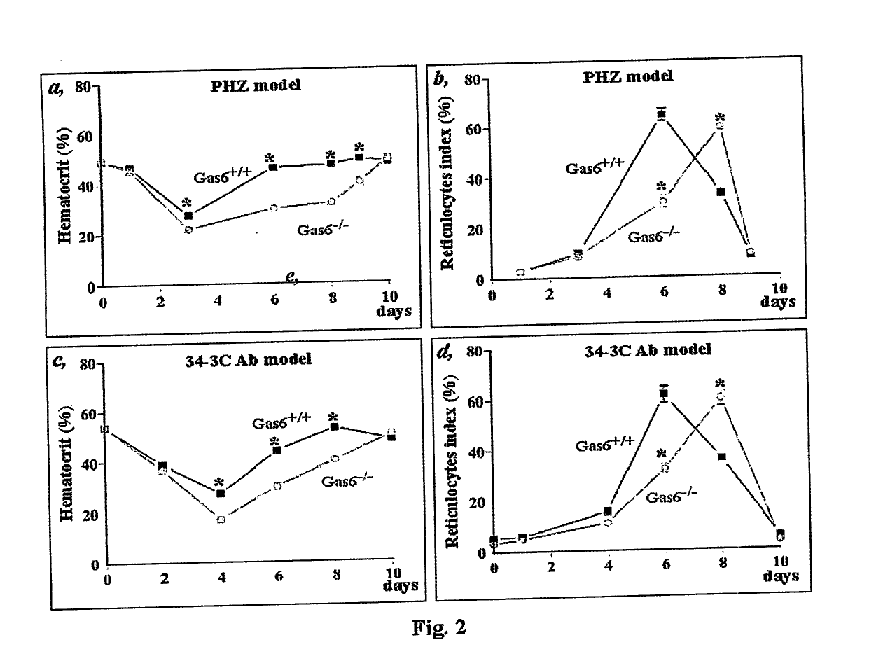

FIG. 2: a,b, Impaired erythropoiesis in Gas6−/− mice in response to phenylhydrazine (PHZ) induced hemolytic anemia. c,d, Impaired erythropoiesis in Gas6−/− mice in response to autoimmune hemolytic anemia, induced by intraperitoneal injection of 34-3C anti-mouse red blood cell monoclonal antibody (200 μg on day 0). Hematocrit (a,c) and reticulocyte index (b,d) levels are expressed as mean±SEM (n=8; *, P<0.05).

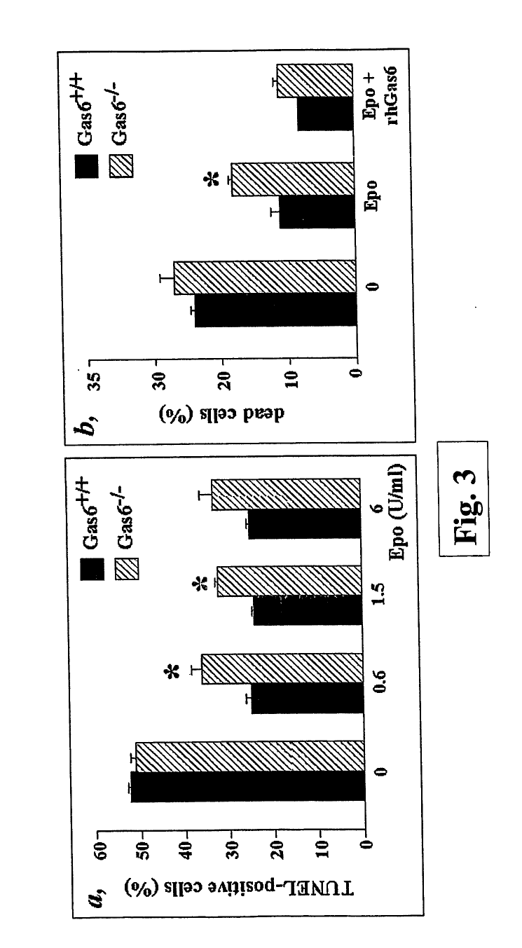

FIG. 3: Impaired Epo-survival response of Gas6−/− erythroblasts. a, TUNEL assay of WT and Gas6−/− nucleated spleen cells on day 6 after PHZ injection. Cells were cultured for 16 h in the presence or absence of recombinant human erythropoietin (Epo) at the indicated concentrations. TUNEL assay was quantfied by flow cytometry (n=8, mean±SEM; *, p<0.05 versus +/+) and results expressed as percent of cells double-stained for TUNEL and Ter-119. b, Dead cell count of nucleated spleen cells (Trypan-blue positive cells) collected on day 6 after PHZ injection and maintained in culture for 24 h. in the presence or absence of Epo (1.5 IU/ml), or Epo plus recombinant human Gas6 (rGas6, 400 ng/ml) (n=8, mean±SEM; *, P<0.05 versus +/+)

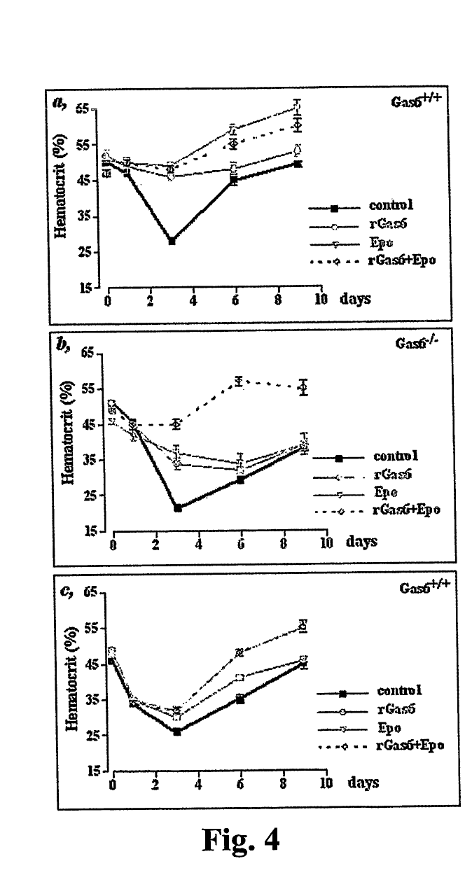

FIG. 4: Gas6 prevents anemia induced by acute hemolysis or blood loss. a,b, Wild type mice (a) and Gas6−/− mice (b) were subjected to PHZ induced hemolytic anemia and treated with saline (control), recombinant human Gas6 (rGas6, 2 μg daily intraperitoneally), recombinant human erythropoietin (Epo, 10 IU every second day intraperitoneally) or a combination of rGas6 and Epo. c, After bleeding (500 μl) on day 0 and 1, wild type mice were treated with saline, Epo, rGas6 or Epo+rGas6 as above, and the erythropoietic response was monitored by determining the hematocrit levels. Hematocrit levels are expressed as mean±SEM (n=6 mice) in all panels.

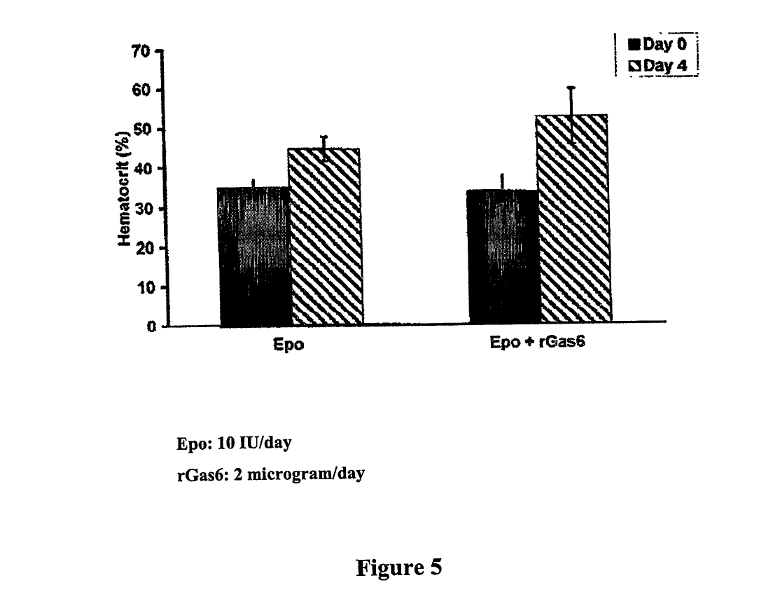

FIG. 5: Effect on hematocrit levels of erythropoietin deficient mice (143.LC, Epo-Tag^{H}) after administration of EPO alone (10 IU/day) or EPO in combination with recombinant Gas6 (EPO: 10 IU/day, rGas6 2 microgram/day). (Filled bars: day 0; striped bars: day 4)

### EXAMPLES

### Example I

### Mice Deficient in Gas6 (Gas6^{-/-} Mice) Have a Decreased Reticulocyte Count

Animal experiments were conducted according to the guiding principles of the American Physiological Society and the International Committee on Thrombosis and Haemostasis (Giles et al. (1987) Thromb Haemost 58, 1078-84).

Blood was collected under general anesthesia from retrobulbar plexus of wild type mice (Gas6^{+/+} mice) or mice in which Gas6 expression was abolished by homologous recombination (Gas6^{-/-} mice) (Angelillo-Scherrer et al. (2001), cited above). Reticulocyte counts were performed on smears of blood that had been stained with New Methylene Blue according to standard protocol (Sigma R4132). At least 1000 red blood cells were counted in each determination.

The data are represented as mean±SEM of n determinations. The significance of differences was determined by unpaired Students'-test. Reticulocytes represented 24.6±4.2% o (n=8) of the circulating red blood cells in Gas6^{+/+} mice and only 13.1±3.1% o (n=8) of the red blood cells in Gas6^{-/-} mice (p<0.001). These data indicate that Gas6 is necessary for maintaining the reticulocyte count within the normal range.

### Example II

### Gas6 Deficient (Gas6^{-/-}) Mice Have Reduced Erythroid Reserves

Single-cell suspensions were obtained from bone marrow and spleen isolated from Gas6^{+/+} and Gas6^{-/-} mice. Ammonium chloride lysis of mature red blood cells was performed. Bone marrow and spleen cells (10⁶) were incubated on ice with rat anti-mouse CD16/CD32 to block nonspecific binding to Fc receptors. Cell suspension was then stained with (PE)-conjugated-anti-Ter-119 antibody (PharMingen) for 30 min. at 4° C. in 100 μl phosphate-buffered saline containing 0.2% BSA. Appropriate isotype control antibodies were used. Cell surface expression of Ter-119 was analyzed in a Becton Dickinson FACScan using CellQuest software.

Ter-119 is expressed by cells of the erythroid lineage, from proerythroblast to mature red blood cells. Ter-119 is expressed by 29±3% (mean±SEM, n=3) of Gas6^{+/+} bone marrow cells versus 17±3% (mean±SEM, n=3) of Gas6^{-/-} bone marrow cells (p<0.05). Similarly, Ter-119 is expressed by 6.0±1.3% (mean±SEM, n=3) of Gas6^{+/+} spleen cells versus 1.9±0.5% (mean±SEM. n=3) of Gas6^{-/-} spleen cells (p<0.05).

### Example II

### The Number of Erythroid Progenitors is Decreased in the Bone Marrow of Mice Deficient in Gas6 (Gas6^{-/-} Mice)

In vitro clonogenic assays for progenitor cells were used to study distinct populations at different stages of development.

Single-cell suspensions were prepared from bone marrow and spleen of adult mice or livers of day 13.5 embryos (E13.5) and counted in the presence of 3% acetic acid to lyse erythrocytes. For this, cell suspensions were mixed with MethoCult M3434 (StemCell Technologies, Vancouver) as described (Neubauer et al. (1998) Cell 93, 397409). Cells were plated in 35 mm dishes and cultured at 37° C., 5% CO2. Colonies, including erythroid burst or blast-forming units (BFU-E, early erythroid progenitor), were scored at day 7. For the final progenitor cell erythroid colony-forming units (CFU-E) assay, cells were cultured in MethoCult 3230 containing 0.2 U/ml recombinant murine erythropoietin (R&D Systems) and colonies were scored at day 3.

Reduced Erythroid Progenitor Cells in Gas6^{-/-} Embryos

Upon macroscopic inspection, Gas6^{-/-} embryos did not appear paler than WT embryos, indicating that they were not severely anemic. Microscopic examination of E13.5 fetal liver sections showed a similar fraction of erythroid cells per section in both genotypes. This was confirmed by flow cytometry, in which the number of Ter-119⁺ erythroblasts per nucleated liver cell was not significantly different in homogenates of fetal livers from Gas6^{-/-} and WT mice. However, the absolute number of nucleated cells per liver was significantly decreased as compared to the livers of E13.5 wild-type (VT) embryos (5.5±0.9×10⁶ versus 11.9±2.8×10⁶ nucleated liver cells/liver in GaseA and WT embryos, respectively; n=6; P<0.002).

To examine why the erythroid reserve, reflected by the absolute number of fetal liver erythroblasts, is reduced in Gas6^{-/-} embryos, erythroid progenitor cells were quantified in the fetal liver, using in vitro clonogenic assays. The number of E13.5 fetal liver eythroid blast-forming units (BFU-E) and erythroid colony-forming units (CFU-E), relative to the total number of nucleated liver cells, was similar in both genotypes (187±12 BFU-E/10⁵ fetal liver cells and 303±50 CFU-E/10⁵ fetal liver cells in WT versus 211±18 BFU-E/10⁵ fetal liver cells and 307±64 CFU-E/10⁵ fetal liver cells in 6^{-/-}; n=6; P=n.s.). However, in absolute terms, Gas6^{-/-} fetal livers contained only half the number of BFU-E and CFU-E as compared to WT fetal livers (FIG. 1a,b). Thus, in the absence of Gas6, the diminished embryonic erythroid reserve may be accounted for by a reduction in erythroid progenitors.

Reduced Erythroid Progenitor Cells in Gas6^{-/-} Adult Mice

It was further examined whether erythroid reserves were also reduced in adult Gas6−/− mice. Hematocrit levels, red blood cell indices and the morphology of peripheral blood and bone marrow erythroid cells were all normal in Gas6^{-/-} mice (hematocrit: 48.8±1.0% in WT mice versus 49.1±3.9% in Gas6^{-/-} mice; n=12; P=n.s.). However, in 6^{-/-} mice, the rate of new red blood cell production was significantly impaired³³ (reticulocyte count: 2.5±0.1% in WT mice versus 1.3±0.1% in Gas6^{-/-} mice; n=8; P<0.002). Clearance of in vivo biotinylated red blood cells was similar in both genotypes (percent biotinylated red blood cells after 30 days: 31±1.3% in WV and 27±1% in Gas6^{-/-} mice, n=6, P=n.s.).

Subsequently, erythroid progenitor cells in the bone marrow and spleen of adult mice were assessed. Compared to WT mice, adult Gas6^{-/-} mice had significantly fewer erythroblasts in both their bone marrow and spleen (FIG. 1c,d). The latter was also microscopically evident by immunostaining histologic sections of spleens with anti-Ter-119 antibody (FIG. 1e,f). Additionally, the diminished number of erythroblasts in the spleens of Gas6^{-/-} was paralleled by a reduction in the weight of spleens from Gas6^{-/-} mice by ˜20% (113±3 mg in 33 WT mice versus 91±15 mg in 31 Gas6^{-/-} mice; P<0.002). To characterize the apparent defect in erythroblast proliferation/differentiation in Gas6^{-/-} mice, the number of distinct populations of erythroid progenitor cells in adult bone marrow was quantified. Three- to four-fold fewer colonies of BFU-E and CFU-E per 105 plated bone marrow cells developed from Gas6−/− versus WT mice (FIG. 1g,h). Thus, in the absence of Gas6, the number of erythroid progenitors was significantly reduced in the adult bone marrow.

### Example IV

### Impaired Recovery after Acute Hemolytic Anemia Of Gas6 Deficient Mice (Gas6^{-/-} Mice)

Anemia was induced in Gas6^{+/+} and Gas6^{-/-} mice by intraperitoneal injection (0.5 or 2 mg/10 g body weight) of freshly prepared phenylhydrazine. Phenyihydrazine hydrochloride (Sigma P6926) was dissolved in PBS at either 10 or 20 mg/ml and the pH was adjusted to pH 7.4 with NaOH. At day 3 following treatment with low dose of PHZ (two doses of 0.5 mg/10 g, 8 hours apart), Gas6−/− mice had a deeper depression in hematocrit (mean±SEM: 29±0.5%, n=5, p<0.001) than Gas6^{+/+} mice (hematocrit, mean±SEM: 36±2%, n=5). In addition, their spleen (site of red cell production) weighed less (mean±SEM: 142±27 mg n=4, p<0.05) compared to Gas6+/+mice (mean±SEM: 254±28 mg, n=4), indicative of impaired erythropoiesis. Moreover, Gas64^{-/-} mice were more susceptible to hemolysis induced by a high dose (2 mg/10 g in one single dose) of PHZ as all Gas6−/− (n=10) mice succumbed to the hemolysis, compared to the 25% mortality rate in Gas6^{+/+} mice (n=10).

Taken together, these data indicate that lack of Gas6 expression increases the susceptibility to acute hemolysis.

### Example V

### Gas6^{-/-} Mice are Resistant to Epo

Serum Epo levels in Gas6^{-/-} mice were measured in steady-state conditions and in response to anemia induced by PHZ. Under baseline conditions, serum Epo protein levels were undetectable in both WT and Gas6^{-/-} mice (<20 mIU/ml). However, at days 3 and 6 after PHZ injection, serum Epo levels were significantly higher in Gas6^{-/-} than in WT mice (53±12 mIU/ml in WT versus 441±120 mIU/ml in Gas6−/− at day 3; 25±0.5 mIU/ml in WT versus 165±50 mIU/ml in Gas6−/− at day 6, n=4-7, P<0.05). Epo receptor (EpoR) expression in spleen lysates was detected by Western immunoblot (Angelillo-Scherrer et al. (2001), cited above). In both WT and Gas6^{-/-} mice, EpoR was barely detectable under baseline conditions, but expression was dramatically increased 3 and 6 days after PHZ injection. While there was some variability, the EpoR levels between the two genotype mice were not significantly different. The data indicates that, in spite of elevated Epo levels and normal EpoR expression, there is an attenuated response to anemia in the Gas6−/− mice. These results thus suggest that the Gas6-deficient mice are resistant to Epo and tried to compensate this defect—in vain however—by elevating their Epo production. Since iron reserves are necessary for a sufficient erythropoiefic response and iron depletion is a common cause of resistance to Epo, the mice were assessed for iron stores. Per's staining of bone marrow cytospins revealed adequate iron stores in both WT and Gas6^{-/-} mice (data not shown). Serum iron levels were also within the normal range (49±2 μm/l in WT and 33±1.7 μm/Il in 6^{-/-}). Thus, the impaired erythropoietic response in anemic Gas6^{-/-} mice, in spite of elevated Epo levels, is not attributable to iron deficiency.

### Example VI

### Gase Erythroblasts are Less Sensitive to Epo And Undergo Increased Cell Death

Spleens and livers were paraformaldehyde-fixed and 7 μm paraffin sections were stained for Ter-119 (BD Biosciences Pharmingen, San Diego, Calif.). Apoptosis was evalutated by Terminal deoxyuridine triphosphate(TdT)-mediated deoxyuridine triphosphate (dUTP)-biotin end (TUNEL)-labeling. ABC Vectastain kit (Vector, Burlingame, Calif.) with DAB-cobalt-nickel staining was used to localize TUNEL-positive cells.

TUNEL-staining of spleen sections from WT and Gas6^{-/-} mice under baseline conditions did not reveal any differences in the extent of apoptosis. However, 3 and 6 days after induction of hemolysis with PHZ, more apoptotic cells were detected in the red pulp of spleens from Gas6^{-/-} mice as compared to those from WT mice. To quantify these apparent differences, nucleated cells were isolated from spleens 6 days after PHZ injection, when 80-90% of the nucleated cells are erythroblasts. When cultured for 16 h in the absence of Epo, approximately 50% of the erythroblasts were apoptotic, with no detectable difference between genotypes. However, the survival response provided by exogenously added recombinant Epo, was markedly different, depending on whether the erythroblasts were derived from WT or Gases mice (FIG. 4a). A concentration of 0.6 IU/ml of Epo decreased the number of apoptotic Gases erythroblasts to 36±2.1% (from 51±1.1% when no Epo was added), which was significantly less protection afforded to WT erythroblasts, where this dose left only 25±1.1% apoptotic erythroblasts (down from 52±0.7% apoptotic cells with no Epo added). Indeed, a 2.5-fold higher concentration of Epo was required to provide Gas6′ erythroblasts similar protection against apoptosis. These findings were further confirmed with Trypan blue staining to assess erythroblast death, showing that the Epo-survival response was impaired in the absence of Gas6. Finally, addition of recombinant Gas6 (rGas6) with exogenous Epo significantly reduced the death of WT and Gas6^{-/-} erythroblasts, indicating that rGas6 fully restored the impaired Epo-survival response of Gas6^{-/-} erythroblasts. These data indicate that lack of Gas6 in erythroblasts prevents an optimal survival response to Epo, and that exogenous soluble rGas6 may rescue the cells from death.

Gas6, Tyro 3, AxI and Mer Expression in Erythroblasts

Assessment of the expression in the spleen of Gas6 receptors was performed under baseline conditions and 6 days after PHZ-induced hemolytic anemia when 80-90% of the splenic cells are erythroblasts (Angelillo-Scherrer et al. (2001), cited above). Tyro 3, AxI and Mer expression were all detectable by Western immunoblot, and without significant differences in intensity between WT and Gas6^{-/-} mice (FIG. 3b). UT7 cells, a human erythroid cell line, were treated with Epo (FIG. 3c). Gas6 was readily detected by Western immunoblot in lysates of quiescent cells. Both cellular and secreted Gas6 expression were augmented by Epo in a dose-dependent manner, providing additional support for a model in which Epo may upregulate and induce the secretion of Gas6.

### Example VII

### Gas6 Prevents Anemia Induced by Hemolysis or Blood Loss

WT and Gas6^{-/-} mice were injected on days 0 and 1 with PHZ, and treated from day 0 with saline i.p. (controls) daily, rGas6 2 μg i.p. daily, recombinant Epo (rEpo) 10 IU i.p. every second day, or both rGas6 and rEpo. Hematocrits and reticulocyte counts were monitored. Since rGas6 has mitogenic and survival effects on several cell types, it was first confirmed that the in vivo clearance of biotinylated red blood cells in both WT and Gas6^{-/-} mice was not affected by rGas6 treatment (percent biotinylated red blood cells after 5 days: 53±3% in WT and 55±7% in Gas6^{-/-} mice; at day 8: 39±2% in WT and 37±2% in Gas6^{-/-} mice; n=6, P=n.s.)

Administration of Epo entirely protected WT mice from PHZ-induced anemia (FIG. 4a), and in fact, resulted in an elevated hematocrit from day 6 onward. Based on our previous findings of higher Epo levels in Gas6^{-/-} mice in spite of a lower hematocrit and reticulocyte response, it was not expectes that Gas6^{-/-} mice would respond to Epo. While over the entire study period, Epo did not have a significant effect (P=0.094), Epo did induce a significant increase in the hematocrit of the Gas6^{-/-} mice at day 3 (P<0.001), and a moderate improvement at days 1 and 6 (P=0.04, P=0.06, respectively) as compared with Gas6^{-/-} saline treated controls (FIG. 4b). But, hematocrit levels remained significantly less than in Epo-treated WT mice at all time points (P<0.001). This suboptimal response to Epo occurred in concert with reduced reticulocyte counts (reticulocyte index: 30±4% in WT versus 1 1±1% in Gas6^{-/-}, n=6, P=0.007 at day 3 and 50±3% in WT versus 20±4% in Gas6^{-/-} at day 6, n=6, P=0.04).

Administration of rGas6 to Gas6^{-/-} mice also provided significant protection against PHZ hemolysis-induced anemia (P=0.003) over the entire study period, with significant benefit specifically observed at days 3 and 6 (P<0.02) when endogenous Epo levels were elevated (FIG. 4b). Notably, at the dose given, rGas6 was also remarkably effective at protecting WT mice from PHZ-induced anemia over the study period (P=0.001), and most significantly at day 3 (P<0.001), largely interfering with the typical early profound decrease in hematocrit (FIG. 4a). While Epo alone, as compared with rGas6 alone, resulted in significantly higher hematocrits in the WT mice at days 3, 6, and 9, rGas6 yielded an essentially normal hematocrit by day 6, and did not cause the hematocrit to rise above the normal pretreatment level, as was seen with Epo (FIG. 4a). Overall, the data indicate that administration of soluble rGas6 enhances erythropoiesis in WT mice during anemia and, importantly, without causing excess erythrocytosis. All mice tolerated the rGas6 treatment without any observable side-effects.

Next it was evaluated whether Epo and rGas6 have a synergistic effect in vivo. In WT mice, no difference was observed between the treatment with Epo alone or Epo+Gas6 (FIG. 5a). When Gas6^{-/-} mice were treated with the combination of rGas6 plus Epo, an obvious synergistic effect of the two agents on erythropoiesis was observed. The erythropoietic activity of Epo was completely restored such that there was a robust increase in the hematocrit from day 3—a significantly better response than with Epo alone (P<0.001)—and reticulocyte counts reached levels observed with similarly treated WT mice (reticulocyte index: 31±5% in WT versus 35±5% in Gas6^{-/-}, n=6, P=n.s. at day 3 and 44±5% in WT versus 55±5% in Gas64^{-/-} at day 6, n=6, P=n.s.) (FIG. 4a, b).

The therapeutic potential of rGas6 was not restricted to the PHZ-induced anemia model. rGas6 also stimulated erythropoiesis in WT mice after bleeding (500 μl day 0 and 1). Indeed, mice receiving rGas6 (2 μg daily) developed significantly less severe anemia than those receiving saline (rGas6 versus saline control: P<0.001 over the 9-day study period), most notably apparent at days 3 and 6 (P<0.005; FIG. 4c). Furthermore, and similar to the effect observed with the PHZ-induced anemia model, treatment with Epo or rGas6 were both effective at day 3, although Epo alone increased the hematocrit levels more than rGas6 alone (P<0.001), specifically evident at days 6 and 9 (FIG. 4c), and ultimately to a hematocrit significantly above normal (P<0.02). Treatment with the combination of rGas6 and Epo provided a response almost identical to that of Epo alone (P=0.61).

### Example VIII

### Treatment of Chronic Anemia with Gas6

Different mice models with chronic anemia have been described. Subtotal nephrectomy in mice results in uremic mice with chronic anemia. They have stable elevations in blood urea nitrogen (mean levels rise from normal range of 26 mg/dl up to a mean of 85 mg/dl) and stable decreases in hematocrit from the normal range (approximately 45%) to approximately 33%. The well-established procedure is performed by surgically removing one kidney, and resecting 2/3 of the other (Hamamori et al. (1995) J Clin Invest. 95, 1808-1813). These mice are known to respond to recombinant or plasma-derived Epo, with persistent increase in hematocrit to levels above normal—up to 68% (Hamamori et al. (1995), cited above). The transgenic mouse strain 134.3LC (Epo-TAg^{H}) displays a severe chronic anemia resembling that observed clinically during chronic renal failure, with a stable chronic anemia due to a relative deficiency in eythropoietin. Mice homozygous for the modified Epo allele have hematocrits in the range of 13-20%, but are otherwise healthy appearing. With administration of exogenous murine Epo, they initially respond with a prominent rise in hematocrit, to levels above normal. Subsequently (after 3-5 weeks of treatment), the response diminishes and their hematocrits fall, as they develop an immune response against endogenous and exogenous Epo (Rinsch et al. (2002) cited above). A third model of a disease associated with chronic anemia is that of mice with beta-thalassemia (Bohl et al. (2000) Blood 95, 2793-2798). These mice have been shown to respond to erythropoietin delivered via gene therapy routes, with hematocrits rising to normal or above normal range, depending on how much Epo is delivered.

Mice have been generated that represent the anemia of chronic renal insufficiency with low level of Epo and a chronic anemia. These mice do respond to exogenous and endogenous Epo. The mice will be provided for study by the lab of Dr. Patrick Maxwell, Imperial College London.

Mice of each of these models with chronic anemia are treated with a range of daily intraperitoneal doses of rGas6 (from 0.2 ug/day to 10 ug/day) and/or recombinant human Epo (Epo) ranging in doses from 0.5 IU to 10 IU every other day intraperitoneally. Experiments are performed over a period of up to 8 weeks. Hematocrits, reticulocyte counts and serum Epo levels are monitored every 2-3 days from blood obtained by retro-orbital puncture.

The same experiments are performed on mice with acute anemia (PHZ-induced hemolytic mice, see example VII).

The minimum dose of rGas6 or Epo required to achieve a stable, normal (or at least target) hematocrit levels is established. It is confirmed that rGas6 does not result in polycythemia in these models, whereas Epo does induce polycythemia.

### Example IX

### Establishment of the Optimal Gas6/Epo Combination

In the previous experiment the minimum doses of recombinant Gas6 and Epo that are individually necessary to attain a normal hematocrit in the different models of anemia. In a next set of experiments, Gas6 and Epo are administered in combinations to these mice.

The minimum doses of Gas6 and Epo that provided full restoration or protection from anemia are first administered in combination. The hematocrit is demonstrated in some cases to rise to above normal under these conditions, due to the effects of Epo. In separate experiments, the amounts of rGas6 and Epo are decreased, identifying different combinations that restore normal hematocrit levels or provide protection, while not inducing polycythemia. It is shown that a combination of doses of Epo and rGas, each below that which would be required for a therapeutic response when given individually, cures or is protective from anemia, while not inducing polycythemia.

### Example X

### Gas6 in the Treatment of Anemia in Mice Resistant To Epo

The transgenic mouse strain 134.3LC (Epo-TAgH) displays a severe chronic anemia resembling that observed clinically during chronic renal failure, with a stable chronic anemia due to a relative deficiency in eythropoietin. Mice homozygous for the modified Epo allele have hematocrits in the range of 13-20%, but are otherwise healthy appearing. With administration of exogenous murine Epo, they initially respond with a prominent rise in hematocrit, to levels above normal. Subsequently (after 3-5 weeks of treatment), the response diminishes and their hematocrits fall, as they develop an immune response against endogenous and exogenous Epo (Rinsch et al. (2002) Kidney Int. 62, 1395-1401.

Transgenic Epo-TAgH mice are treated with daily intraperitoneal doses of rGas6 (from 0.2 ug/day to 10 ug/day) or recombinant human Epo (Epo) ranging in doses from 0.5 IU to 10 IU every other day intraperitoneally.

In the Epo-treated Epo-TAgH mice, an initial rise in hematocrit is observed, with levels above normal, followed by a gradual decrease in hematocrit.

In the Gas6 treated mice, a gradual rise in hematocrit is observed up to a normal level, which remains stable up to 12 weeks after initiation of the therapy.

### Example XI

### Effect of Epo Alone or in Combination with Recombinant Gas60N Chronic Anemia

Patients suffering from chronic renal failure develop anemia due to inadequate erythropoietin (Epo) production by the kidney. Recombinant Epo, in some patients, partially restores hematocrit and blood hemoglobin concentration, decreasing the need for blood transfusions. Epo alone is not always effective.

Animal models using partial nephrectomy have been developed to reproduce conditions that lead to anemia in patients with chronic renal failure. Nevertheless, these models suffer from shortcomings, including hematocrits that do not decrease to the levels observed in chronic renal failure and toxicity due to renal failure conducting animals to death (Hamamori, Y. et al. 1995, J Clin Invest, 95:1149-1162).

A transgenic mouse strain (134.3 LC, Epo-Tag^{H}), which has a stable chronic anemia due to deficiency of erythropoietin, was used [Maxwell et al. (1993) Kidney Int 44, 1149-1162]. This mouse model constitutes a valuable tool for refining gene transfer approaches for delivery of Epo. Mice homozygous for the modified Epo allele are severely anemic, with hematocrits ranging from 13% to 25%, but otherwise appear healthy and remain active. Heterozygous mice are moderately anemic with hematocrit levels below 45% but higher than 25%. Homozygous mice display hematocrit levels comparable to those observed in patients with chronic renal failure.

Mice that are heterozygous for Epo-Tag^{H}, were treated with either Epo alone (10 IU/day) or Gas6 alone or with Epo 10 IU/day plus recombinant Gas6 2 μg per day (see FIG. 5). When mice were injected with 2 μg of rGas6 alone per day, a dose that was shown to be effective in restoring hematocrit in acute anemia models, we did not observe an effect after 8 days of treatment. We therefore increased the dose of rGas6 to 4 microgram per day and then obtained a significant increase in hematocrit already after 4 days. Co-administration of rGas6 2 μg per day with Epo 5 IU/day resulted in a slightly higher hematocrit over that of either drug administered alone. The synergistic benefical effect became significant when low dose rGas6 (2 microgram per day) was administered together with Epo (10 IU per day), resulting in a rise in the hematocrit higher than Epo alone or rGas6 alone. Thus, Gas6 is effective in chronic anemia, augments the effects of Epo, and may have Epo dose-sparing effects.

These results indicate that the addition of Gas6 to Epo, for treatment of anemia, has a greater beneficial effect over Epo alone. These results also indicate that combination therapy with Gas6+Epo will be therapeutically beneficial in the treatment of patients with anemia.

### Example XII

### Gas6 in the Treatment of Anemia in Patients Resistant to Epo

A subgroup of anemic patients suffering from chronic renal failure, which do not respond to Epo are treated with an effective dose of Gas6 for 4 weeks. At the end of this time period it is established that the hemoglobin level of these patients has normalized.

1-20. (canceled)

21. A method for the treatment, curing or prevention of anemia in a patient, which comprises administering to said patient a combination of a Gas6 compound or a DNA sequence encoding a Gas6 compound and erythropoeitin, or an analogue, mutant, variant or derivative of said erythropoeitin, or a physiologically tolerated salt of said EPO derivative, either simultaneously or sequentially, thereby ensuring a synergistic rescue effect on erythropoeisis in said patient.

22. The method according to claim 21, wherein said Gas6 compound is a Gas6 protein or an analogue, mutant, variant or derivative thereof, or a physiologically tolerated salt of said Gas6 derivative.

23. The method of claim 21, which comprises administering a portion of an effective amount of said erythropoeitin, or an analogue, mutant, variant or derivative of said erythropoeitin, or a physiologically tolerated salt of said EPO derivative, normally required to treat anemia in said patient, thereby ensuring a reduced risk of adverse side-effects.

24. The method of claim 23, which comprises administering 5 to 90% of the amount of said said erythropoeitin, or an analogue, mutant, variant or derivative of said erythropoeitin, or a physiologically tolerated salt of said EPO derivative, normally required to treat anemia in said patient.

25. The method of claim 23, wherein said patient is susceptible to the side-effects of Epo and for which treatment with Epo is contra-indicated.

26. The method of claim 24, wherein said patient susceptible to the adverse side-effects of Epo is a patient suffering from hypertension.

27. The method of claim 21, wherein said patient is a patient which has an inadequate Epo response.

28. The method according to claim 21, wherein said DNA encoding a Gas6 compound is comprised in a vector.

29. The method according to claim 21, wherein said DNA encoding a Gas6 compound is engineered into a cell.

30. The method of claim 21, wherein said anemia is caused by or associated with a disorder selected from the group consisting of chronic renal failure, aplastic anemia, hemolytic anemia, malignancy, an endocrine deficiency or is the result of chemotherapy.
